(19) United States
(12) Patent Application Publication
(10) Pub. No.: US 2009/0186818 A1
(43) Pub. Date: Jul. 23, 2009

(54) Therapeutic Method for Glycaemic Control

(76) Inventor: Anker G. Lundemose, Oxford (GB)

Correspondence Address: OSI PHARMACEUTICALS, INC., 41 PINELAWN ROAD, MELVILLE, NY, 11747, US

(21) Appl. No.: 12/296,716

(22) PCT Filed: Apr. 10, 2007
(86) PCT No.: PCT/GB2007/050188

§ 371 Date: Dec. 2, 2008

(30) Foreign Application Priority Data
Apr. 10, 2006 (GB) 0607153.4

(51) Int. Cl. A61K 38/17; A61K 31/426; A61P 3/10

(52) U.S. Cl. 514/12; 514/365

(57) ABSTRACT

The invention relates to a therapeutic method for glycaemic control, comprising the use of a combination of a DPIV inhibitor and a GLP-1 receptor agonist.

### FIELD OF THE INVENTION

This invention relates to a therapeutic method for glycaemic control, in particular to a method for the treatment of type 2 diabetes.

### BACKGROUND ART

Diabetes mellitus is a chronic metabolic disorder characterized by the presence of hyperglycaemia (raised blood glucose concentrations). It may be divided into four general subclasses, including i) type 1 or insulin-dependent diabetes mellitus (IDDM) (caused by beta-cell destruction and characterized by absolute insulin deficiency), ii) type 2 or non-insulin-dependent diabetes mellitus (NIDDM) (characterized by impaired insulin action and impaired insulin secretion), iii) other specific types of diabetes (associated with various identifiable clinical conditions or syndromes such as genetic defects of beta-cell function e.g. maturity-onset diabetes of the young types 1-3 and point mutations in mitochondrial DNA), and iv) gestational diabetes mellitus.

The prevalence of type 2 diabetes is high and is growing at an alarming rate. The global burden of diabetes mellitus is expected to reach 300 million by the year 2025, with more than 90% of these individuals having type 2 diabetes.

The predominant pathophysiological defects leading to hyperglycaemia in type 2 diabetes are impaired insulin action (insulin resistance) and impaired insulin secretion (beta-cell dysfunction). Treating hyperglycaemia is therapeutically important in diabetes mellitus in order to prevent symptoms caused by the raised blood glucose concentrations, such as polyuria (excessive urination) and polydipsia (excessive thirst), and to reduce the risk of diabetic complications. The chronic hyperglycaemia of diabetes mellitus is associated with significant, often devastating long-term complications in the eyes, kidneys, nerves and blood vessels. The largest study of pharmacotherapy in type 2 diabetes, The United Kingdom Prospective Diabetes Study (UKPDS), demonstrated that lowering blood glucose concentrations with pharmacotherapy in type 2 diabetes reduces the risk of complications. [Lancet 352:837-853, 1998]. The study showed that there was no lower threshold for the benefits of glucose lowering and that any additional glucose lowering would further reduce the risk of development of diabetic complications.

The UKPDS also demonstrated that an inexorable decline in beta-cell function occurs with time in type 2 diabetes [Diabetes 44:1249-1258, 1995]. This leads, in the majority of patients, to worsening of glycaemic control with time, requiring addition of more and more therapies as the disease progresses.

There are a number of oral agents currently available to treat type 2 diabetes. The three classes of agent which are most commonly prescribed are metformin, the sulphonylureas and the thiazolidinediones (TZDs). Metformin acts predominantly by decreasing glucose output from the liver, it is associated with gastrointestinal side-effects in many patients and has no impact on the decline in beta-cell function with time. The sulphonylureas act by increasing insulin secretion, are associated with the side effects of weight gain and hypoglycaemia (low blood glucose concentrations) and, like metformin, have no impact on the decline in beta-cell function with time (see UKPDS). The TZDs act as insulin sensitizers and, whilst they are the only class of oral agent currently licensed to treat type 2 diabetes which decreases the rate of decline of beta-cell function with time, they are associated with the side effects of weight gain and oedema.

Glucagon-like peptide 1 (GLP-1) is a 30/31 amino acid polypeptide which is released from intestinal L-cells after feeding. Its properties and function are discussed at length in Deacon, Diabetes, 53, 2181-2189, 2004 and D'Alessio and Vahl, Am. J. Physiol. Endocrinol. Metab., 286, E882-890, 2004. In summary, GLP-1 is an insulinotropic hormone and plays a role in the incretin effect, the raised insulin response observed when glucose is adsorbed through the gut. More generally GLP-1 seems to have a broad role as a mediator of post-prandial glucose homeostatis and in particular appears to regulate a number of processes that reduce blood glucose fluctuations such as gastric emptying, glucagon secretion and food intake. It has also recently been suggested that GLP-1 is involved in the regulation of β-cell mass, since proliferation and differentiation of new β-cells in response to GLP-1 has been observed. A specific GLP-1 receptor has been cloned from pancreatic islet cells and is the only known mediator of GLP-1 signalling in mammals. The GLP-1 receptor is a member of the secretin-VIP family of seven membrane-spanning G-protein coupled receptors. Endogenous ligand binding is highly specific and there is little affinity for glucagon or glucose dependent insulinotropic polypeptide (GIP), another gastrointestinal incretin hormone. A hallmark of the insulinotropic effect of GLP-1 is the dependence on glucose levels, since GLP-1 has only minimal activity on insulin secretion when glucose concentrations are normal. β-cell signaling through the GLP-1 receptor is therefore distinguished from other insulin secretagogues, such as sulfonylureas, which induce insulin secretion independently of the glucose concentration. Administration of GLP-1 to patients with type-2 diabetes has proved effective in lowering their blood glucose level and due to the glucose dependency of its anti-hyperglycaemic effect there appears to be little risk of hypoglycaemia with such an approach.

The above mentioned glucoregulatory actions of GLP-1 make it an attractive prospect for the treatment of type 2 diabetes. However its therapeutic potential is limited because it is rapidly degraded by dipeptidyl peptidase IV (DPIV). This, together with its renal clearance, means it has a half life of less than 2 minutes. DPIV is a membrane-bound ectoenzyme which is found in numerous sites, including the kidney, intestine and capillary endothelium. DPIV cleaves an N-terminal dipeptide from peptides (such as GLP-1) which have as the penultimate residue the amino acid Pro or Ala. It has also been speculated that GLP-1 is degraded by the actions of the enzyme neutral endopeptidase (NEP) 24.11. NEP 24.11 is a membrane bound zinc metalloprotease which cleaves peptides at the N-terminal side of aromatic or hydrophobic amino acids. 6 potential NEP 24.11 cleavage sites in GLP-1 have been identified.

Therefore a number of approaches to produce more useful GLP-1 receptor agonists have been attempted. One particular class of GLP-1 receptor agonist of interest is the class of GLP-1 mimetics having extended half lives relative to GLP-1 itself. Approaches to extending the half-life relative to GLP-1 have included replacing the penultimate residue with an amino acid other than Ala or Pro thereby to reduce or eliminate the susceptibility to DPIV degradation. Another approach has been to extend the C-terminus which apparently makes it a poorer substrate for NEP 24.11.

One longer acting GLP-1 receptor agonist is exendin-4 which was originally isolated from the venom of the Gila monster. Its synthetic analogue, known as exenatide (AC2993, Byetta™, Amylin Pharmaceuticals, San Diego, USA), has been extensively tested in humans, showing a half life of approximately 2.4 hours. Exendin-4 (exenatide) has 53% sequence identity with native GLP-1 and is DPIV resistant since its penultimate residue at the N-terminus is Gly. It is also a poor substrate for NEP 24.11.

Another mimetic is liraglutide (NN2211, Novo Nordisk, Copenhagen, Denmark) which has 97% sequence identity to native GLP-1. Acylation with a fatty acid chain in liraglutide promotes binding to albumin, thereby reducing access to the N-terminus by DPIV, and allowing the molecule to escape renal filtration. Liraglutide has a half life of approximately 12 hours in humans.

A further example is CJC-1131 (ConjuChem, Montreal, Canada) in which GLP-1 has been conjugated through a covalent linker to albumin. This compound has a half life of around 2 weeks in humans.

Another example of a GLP-1 mimetic is ZP-10 (Zealand Pharma, Glostrup, Denmark).

GLP-1 and its mimetics by virtue of their proteinaceous nature are typically administered by injection, for example exenatide (Byetta™) is indicated for bidaily injection.

From the available data, protease-resistant GLP-1 analogues appear to be associated with relatively few side effects other than notable effects on the gastrointestinal tract. However nausea and vomiting are commonly reported adverse reactions which have a serious impact on patient's comfort and compliance. For example, the most dominant side effect of exenatide is nausea, which is seen in up to 57% of patients (Heine et al, Abstract 9, ADA, 65th Scientific Sessions, 2005).

It is known that DPIV inhibitors may be useful for the treatment of impaired glucose tolerance and diabetes mellitus, see International Patent Application No. WO97/40832. Clinical data suggest that the glucose lowering that occurs in response to treatment with DPIV inhibitors is not accompanied by significant side effects such as weight gain and hypoglycaemia. In addition, pre-clinical data exist which suggest that DPIV inhibitors preserve beta-cell mass, see International Patent Application No. WO01/72290, raising the possibility that treatment with a DPIV inhibitor would decrease the rate of decline of beta-cell function that occurs with time in type 2 diabetes.

There is a need to find new and improved regimens for the treatment of type 2 diabetes. The present invention provides a novel combination therapy for the treatment of type 2 diabetes. This method has the potential to provide greater efficacy than current therapies given alone without introducing any side-effect liability.

The combination of active ingredients for the treatment of type 2 diabetes has been suggested, see e.g. International Patent Application No. WO2006/000567. However, the specific combination of a DPIV inhibitor and a GLP-1 agonist, in particular in a dosage regimen which limits the side-effects of the therapy has not been suggested.

### SUMMARY OF THE INVENTION

The present invention provides the use of a combination of a DPIV inhibitor and a GLP-1 receptor agonist for glycaemic control, e.g. in the treatment of type 2 diabetes.

### DETAILED DESCRIPTION OF THE INVENTION

The present invention provides a method for the treatment of type 2 diabetes in a mammal, such as a human, which method comprises administering a combination of a DPIV inhibitor and a GLP-1 receptor agonist, to a mammal in need thereof.

The present invention provides the use of a combination of a DPIV inhibitor and a GLP-1 receptor agonist for treatment of type 2 diabetes.

The invention also provides the use of a DPIV inhibitor in the manufacture of a medicament for use in combination with a GLP-1 receptor agonist, for the treatment of type 2 diabetes.

The invention also provides the use of a GLP-1 receptor agonist in the manufacture of a medicament for use in combination with a DPIV inhibitor, for the treatment of type 2 diabetes.

The invention also provides the use of a GLP-1 receptor agonist in combination with a DPIV inhibitor in the manufacture of a medicament for the treatment of type 2 diabetes.

The invention also provides a kit comprising (i) a GLP-1 receptor agonist; (ii) a DPIV inhibitor and (iii) instructions directing use of the GLP-1 receptor agonist and the DPIV inhibitor in combination for the treatment of type 2 diabetes in a mammal, such as a human.

The combination therapy according to the invention may be administered as a first-line therapy.

First-line therapy is defined as the first course of pharmaceutical treatment used against a disease, thus in the present case it represents the first pharmacological intervention to treat type 2 diabetes in a patient diagnosed as having inadequate glycaemic control. In a type 2 diabetic patient this will generally be a patient whose hyperglycaemia can no longer be controlled satisfactorily by diet, weight reduction and/or exercise alone. The combination therapy of the invention may be used to treat a human that has failed to achieve adequate glycaemic control using diet, weight reduction and/or exercise alone. Such a patient population has not previously been treated with a first line combination therapy comprising a DPIV inhibitor and a GLP-1 receptor agonist.

The present invention also provides a method for the treatment of type 2 diabetes in a mammal, such as a human, which method comprises administering a combination of a DPIV inhibitor and a GLP-1 receptor agonist as first-line therapy, to a mammal in need thereof.

The invention also provides the use of combination of a DPIV inhibitor and a GLP-1 receptor agonist as first-line therapy for the treatment of type 2 diabetes.

The invention also provides the use of a DPIV inhibitor in the manufacture of a medicament for use in combination with a GLP-1 receptor agonist as first-line therapy, for the treatment of type 2 diabetes.

In the methods of the invention type 2 diabetes is thereby treated.

By combining the GLP-1 receptor agonist with a DPIV inhibitor it becomes possible to reduce the dose or frequency of administration of the GLP-1 receptor agonist compared to the situation where the GLP-1 receptor agonist is not administered in combination with a DPIV inhibitor (e.g. to once daily dosing) whilst still achieving effective glycaemic control.

The invention provides significant advantages over other therapies for glycaemic control, including the possibility for:
reduced risks of side effects, e.g. nausea and vomiting, associated with GLP-1 receptor agonists, potentially resulting in better patient compliance;
fewer injections of GLP-1 receptor agonists, thereby increasing patient comfort;
greater blood glucose lowering than with monotherapy; and/or
maximisation of the opportunity for early positioning in the treatment continuum of a therapy which reduces decline in beta-cell function.

In accordance with the invention coadministration of the DPIV inhibitor and a GLP-1 receptor agonist includes administration of a formulation which includes both the DPIV inhibitor and GLP-1 receptor agonist, or the essentially simultaneous, sequential or separate administration of separate formulations of the DPIV inhibitor and GLP-1 receptor agonist.

In one embodiment of the present invention the separate formulations of the DPIV inhibitor and a GLP-receptor agonist are co-administered essentially simultaneously. In another embodiment of the present invention the separate formulations of the DPIV inhibitor and a GLP-receptor agonist are co-administered separately in time.

In accordance with the invention the coadministration is preferably administration of separate formulations of the DPIV inhibitor and GLP-1 receptor agonist.

Typically the DPIV inhibitor is administered 1-3 times per day at or before mealtimes. Typically the GLP-1 receptor agonist is administered 1-2 times per day.

One particular dosage regimen which may be mentioned would be administration of a DPIV inhibitor during the day, such as prandial administration with meals such as the first and/or second meals of the day (e.g. breakfast and lunch) followed by administration of a GLP-1 receptor agonist with the final meal of the day or before bedtime.

Without being limited by theory, such a regimen could be advantageous since the sparing of endogenous GLP-1 through DPIV inhibition by day is complemented by the supply of exogenous GLP-1 receptor agonist by night.

The DPIV inhibitor for use in the method of the invention is preferably a small molecule DPIV inhibitor. DPIV inhibitors are preferably selective inhibitors e.g. have inhibitory activity which is greater for DPIV than for other DP enzymes. As referred to in the present application “DPIV inhibitor” includes any pharmaceutically acceptable salts of DPIV inhibitors.

Examples of DPIV inhibitors include compounds disclosed in the following patent applications: WO95/15309, WO95/29691, WO98/18763, WO98/19998, WO99/25719, WO99/38501, WO99/46272, WO99/61431, WO99/62914, WO99/67278, WO99/67279, WO00/34241, WO01/34594, WO01/40180, WO01/55105, WO01/52825, WO01/68603, WO01/72290, WO01/81304, WO01/81337, WO01/96295, WO01/97808, WO02/02560, WO02/08090, WO02/14271, WO02/30890, WO02/30891, WO02/38541, WO02/51836, WO02/53548, WO02/62764, WO02/66627, WO02/67918, WO02/68420, WO02/76450, WO02/83109, WO02/83128, WO03/00181, WO03/00181, WO03/00250, WO03/02530, WO03/02531, WO03/02553, WO03/02942, WO03/03250, WO03/03727, WO03/04496, WO03/04498, WO03/104229, WO03/24965, WO03/35057, WO03/35067, WO03/04498, WO03/33524, WO03/33671, WO03/37327, WO03/55881, WO03/57144, WO03/57200, WO03/57666, WO03/68748, WO03/68757, WO03/74500, WO03/84940, WO03/92605, WO03/101449, WO03/101958, WO03/106456, WO04/16587, WO04/16840, WO04/18468, WO04/18469, WO04/24184, WO04/26822, WO04/33455, WO04/37169, WO04/37181, WO04/41795, WO04/43940, WO04/46106, WO04/46148, WO04/48352, WO04/50022, WO04/50658, WO04/52850, WO04/103276, WO04/112701, WO05/12249, WO05/12308, WO05/12312, WO05/16911, WO05/19168, WO05/25554, WO05/26148, WO05/30751, WO05/37779, WO05/37828, WO05/40095, WO05/42533, WO05/42488, WO05/44195, WO05/47297, WO05/51949, WO05/51950, WO05/56541, WO05/56013, WO05/56003, WO05/58849, WO05/58901, WO05/63750, WO05/73186, WO05/75421, WO05/75426, WO05/77900, WO05/82906, WO05/82847, WO05/82349, WO05/82348, WO05/85246, WO05/87774, WO05/87235, WO05/95343, WO05/95381, WO05/95339, WO05/97798, WO05/100334, WO05/108382, WO05/113510, WO05/116012, WO05/11614, WO05/11629, WO05/118555, WO05/120494, EP1245568, EP1258476, EP1258480, EP1338595, JP2002265439 and JP2003300977.

For the avoidance of doubt, the examples disclosed in each of the above mentioned publications are specifically incorporated herein by reference in their entirety, as individually disclosed compounds, especially concerning their structure, use and synthesis.

Examples of specific DPIV inhibitors include sitagliptin, vildagliptin, saxagliptin, denagliptin and alogliptin and salts thereof.

A preferred DPIV inhibitor for use in the method of the invention is glutaminyl thiazolidine or a pharmaceutically acceptable salt thereof, e.g. the hydrochloride salt, see International Patent Application No. WO03/072556.

Preferred embodiments of the invention thus include:
a method for the treatment of type 2 diabetes in a mammal which method comprises administering a combination of glutaminyl thiazolidine or a pharmaceutically acceptable salt thereof, and GLP-1, a GLP-1 mimetic or a GLP-1 receptor agonist;
a method for the treatment of type 2 diabetes in a mammal which method comprises administering a combination of glutaminyl thiazolidine or a pharmaceutically acceptable salt thereof, and a GLP-1 receptor agonist, to a human in need thereof; and
combinations as described above for first line therapy.

GLP-1 receptor agonists include GLP-1 (natural or synthetic), GLP-1 mimetics (including longer acting analogues which are resistant to or have reduced susceptibility to degradation by DPIV and/or NEP 24.11) and other substances (whether peptidic or non-peptidic e.g. “small molecule”) which promote signalling through the GLP-1 receptor. Suitable GLP-1 mimetics include exenatide, liraglutide and CJC-1131. A further example is ZP-10.

GLP-1 mimetics will typically have significant sequence identity to GLP-1 (e.g. greater than 50%, 75, 90 or 95% identity) and may be derivatised e.g. by conjugation to other proteins (e.g. albumin) or through chemical modification.

In one embodiment of the invention the GLP-1 receptor agonist is GLP-1. In another embodiment of the invention the GLP-1 receptor agonist is a GLP-1 mimetic. In another embodiment of the invention the GLP-1 receptor agonist is another substance (whether peptidic or non-peptidic e.g. “small molecule”) which promotes signalling through the GLP-1 receptor.

The DPIV inhibitor and a GLP-1 receptor agonist are each administered in a pharmaceutically acceptable form, including pharmaceutically acceptable derivatives such as pharmaceutically acceptable salts, esters and solvates thereof, as appropriate of the relevant pharmaceutically active agent. In certain instances herein the names used for the active agent may relate to a particular pharmaceutical form of the relevant active agent. It will be understood that the use of all pharmaceutically acceptable forms of the active agents per se is encompassed by this invention.

Pharmaceutically acceptable salts of the preferred DPIV inhibitor glutaminyl thiazolidine include acid addition salts, i.e. where the amino acid basic side chain is protonated with an inorganic or organic acid. Representative organic and inorganic acids include hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic, trifluoroacetic, sulfinic and 3,5-di-tert-butylbenzoic acid. Preferred acid addition salts include the fumarate, benzoate, maleinate, oxalate, 3,5-di-tertiary-butylbenzoate, salicylate, acetate and hydrochloride salts, especially the hydrochloride salt.

The DPIV inhibitor is preferably administered orally. In particular it is preferably formulated in unit doses for administration once, twice or three times a day. When the DPIV inhibitor is glutaminyl thiazolidine or a salt thereof it is preferably administered two or three times a day, e.g. at meal times.

When the GLP-1 receptor agonist is GLP-1 or a GLP-1 mimetic, it is suitably administered by injection e.g. subcutaneous injection.

To prepare the pharmaceutical compositions for use in the methods of the invention the active agents are intimately admixed with a pharmaceutical carrier according to conventional pharmaceutical compounding techniques, which carrier may take a wide variety of forms depending of the form of preparation desired for administration, e.g. oral or parenteral such as intramuscular. In preparing compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included.

Injectable suspensions may also be prepared, in which case appropriate liquid carriers, suspending agents and the like may be employed. The pharmaceutical compositions herein will contain, per dosage unit, e.g. tablet, capsule, powder, injection, teaspoonful and the like, an amount of the active agent necessary to deliver a therapeutically effective amount. The dosages, however, may be varied depending upon the requirement of the patients, the severity of the condition being treated and the compound being employed. The use of either daily administration or post-periodic dosing may be employed.

Preferably these compositions are in unit dosage form such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. For preparing solid compositions such as tablets, the principal active agent is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of the active agent(s). When referring to these preformulation compositions as homogeneous, it is meant that the active agent is dispersed evenly throughout the composition so that the composition may be readily subdivided into equally effective dosage forms such as tablets, pills and capsules. This solid preformulation composition is then subdivided into unit dosage forms of the type described above.

Tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

This liquid forms in which the DPIV inhibitor may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinylpyrrolidone or gelatin.

The method of treating type 2 diabetes as described in the present invention may also be carried out using a pharmaceutical composition comprising a DPIV inhibitor, optionally in combination with and GLP-1, a GLP-1 mimetic or a GLP-1 receptor agonist, and a pharmaceutically acceptable carrier. The pharmaceutical composition may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

For instance, for oral administration in the form of a tablet or capsule, the active agents can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or betalactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitable flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The DPIV inhibitor and a GLP-1 receptor agonist may be administered in any of the foregoing compositions and according to dosage regimens whenever glycaemic control is required. The active agents may be administered on a regimen of 1 to 4 times per day.

Optimal dosages to be administered may be readily determined by those skilled in the art, and will vary with the particular active agent used, the mode of administration, the strength of the preparation, the mode of administration, and the advancement of disease condition. In addition, factors associated with the particular patient being treated, including patient age, weight, diet and time of administration, will result in the need to adjust dosages.

Suitable dosages, including especially unit dosages, of the active agents to be used in the method of the invention include the known dosages including unit doses for these compounds as described or referred to in reference text such as the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein) or the above mentioned publications.

Suitable doses of DPIV inhibitors include those described in the relevant publications mentioned above. Suitable unit doses of the preferred DPIV inhibitor glutaminyl thiazolidine are in the range 10 to 500 mg, e.g. 50, 100, 200 or 300 mg, which unit doses may be administered up to three times a day.

Suitable doses of GLP-1 receptor agonists may also be determined by a person skilled in the art. For example, Byetta™ (exenatide) is dosed at 5 or 10 mcg twice daily. The normal treatment protocol involves subcutaneous injection once before morning and evening meals. Liraglutide is typically administered at 0.045-0.75 mg e.g. 0.45 or 0.75 mg once daily e.g. by subcutaneous injection. As noted above, the dosage or frequency of the above GLP-1 receptor agonists may be reduced when employed in the combination with a DPIV inhibitor in the method according to the invention.

Also, the dosages of each particular active agent in any given composition can as required vary within a range of doses known to be required in respect of accepted dosage regimens for that compound. Dosages of each active agent can also be adapted as required to take into account advantageous effects of combining the agents as mentioned herein. Thus the invention may allow glycaemic control to be achieved using doses of the GLP-1 receptor agonist below those required when such compounds are administered without a DPIV inhibitor. The use of lower doses of such compounds may reduce unwanted side effects such as nausea and hence improving patient compliance.

Suitably, the particularly beneficial effect on glycaemic control in the treatment of type 2 diabetes provided by the method of the invention is an improved therapeutic ratio for the combination of the invention relative to the therapeutic ratio for one compound of the combination when used alone and at a dose providing an equivalent efficacy to the combination of the invention.

The method of the invention may also form part of a triple therapy whereby an additional anti-diabetic agent is also administered to the mammal. A specifically preferred additional antidiabetic agent is metformin. Reference to “metformin” includes any pharmaceutically acceptable salt of metformin e.g. the hydrochloride salt. A suitable daily dosage of metformin is between 50 and 3000 mg, for example 250, 500 mg, 850 mg or 1000 mg. The additional antidiabetic agent e.g. metformin, may be administered simultaneously, sequentially or separately from the DPIV inhibitor and the GLP-1 receptor agonist. In one specific embodiment of this aspect of the invention the metformin and DPIV inhibitor may be administered together as a fixed dose formulation.

Glycaemic control may be characterised using conventional methods, for example by measurement of a typically used index of glycaemic control such as fasting plasma glucose or glycosylated haemoglobin (HbA1c). Such indices are determined using standard methodology, for example those described in: Tuescher A, Richterich, P., Schweiz. med. Wschr. 101 (1971), 345 and 390 and Frank P., ‘Monitoring the Diabetic Patent with Glycosolated Hemoglobin Measurements’, Clinical Products 1988.

The dosage level of each of the active agents when used in accordance with the method of the invention may be less than would have been required from a purely additive effect upon glycaemic control.

The term “therapeutically effective amount” as used herein, means that amount of active compound or pharmaceutical agent that elicits the biological or medicinal response in a tissue system, animal or human, being sought by a researcher, veterinarian, medical doctor or other clinician, which includes alleviation of the symptoms of the disease or disorder being treated.

All publications, including, but not limited to, patents and patent application cited in this specification, are herein incorporated by reference as if each individual publication were specifically and individually indicated to be incorporated by reference herein as fully set forth.

### EXAMPLE

In one treatment group, the comparator group, subjects are treated with exenatide, 5 or 10 mcg, twice daily by subcutaneous injection.

In another treatment group, the group according to the invention, subjects are treated with exenatide, 5 or 10 mcg, once daily by subcutaneous injection, and a DPIV inhibitor once daily by oral administration. The exenatide is administered before bedtime and the DPIV inhibitor is administered before the morning meal.

1. A method for the treatment of type 2 diabetes in a mammal which method comprises administering a combination of a DPIV inhibitor and a GLP-1 receptor agonist, to a mammal in need thereof.

2. The method according to claim 1 which comprises administering a combination of a DPIV inhibitor and a GLP-1 receptor agonist as first-line therapy, to a mammal in need thereof.

3. The method according to claim 1 wherein the mammal is a human.

4. The method according to claim 3 wherein the human has failed to achieve adequate glycaemic control using diet, weight reduction and/or exercise alone.

5. The method according to claim 1 wherein the DPIV inhibitor and GLP-1 receptor agonist are administered simultaneously, sequentially or separately.

6. The method according to claim 5 wherein the DPIV inhibitor and GLP-1 receptor agonist are administered separately.

7. The method according to claim 1 wherein the DPIV inhibitor is administered orally.

8. The method according to claim 1 wherein the DPIV inhibitor is glutaminyl thiazolidine or a pharmaceutically acceptable salt thereof.

9. The method according to claim 8 wherein the DPIV inhibitor is glutaminyl thiazolidine hydrochloride.

10. The method according to claim 1 wherein the GLP-1 receptor agonist is a GLP-mimetic.

11. The method according to claim 10 wherein the GLP-mimetic is exenetide, liraglutide, CJC-1131 or ZP-10.

12. The method according to claim 1, wherein the dose or frequency of administration of the GLP-1 receptor agonist is reduced compared to the situation where the GLP-1 receptor agonist is not administered in combination with a DPIV inhibitor, thereby reducing the risks of side effects associated with administration of the GLP-1 receptor agonist.

13. The method according to claim 1 wherein the GLP-1 agonist is administered once daily.

14. The method according to claim 1 wherein the DPIV inhibitor is administered one or more times during the day and the GLP-1 receptor agonist is administered with the final meal of the day or before bedtime.

15. The method according to claim 1 which additionally comprises administering metformin to the mammal.

16. A kit comprising (i) a GLP-1 receptor agonist; (ii) a DPIV inhibitor and (iii) instructions directing use of the GLP-1 receptor agonist and the DPIV inhibitor in combination for the treatment of type 2 diabetes in a mammal.
